(19) United States
(12) Patent Application Publication
(10) Pub. No.: US 2024/0336792 A1
(43) Pub. Date: Oct. 10, 2024

(54) METHOD FOR MANUFACTURING STERILE BIO-INK

(71) Applicant: 3D GLOBAL BIOTECH INC., New Taipei City (TW)

(72) Inventors: Keng-Liang Ou, Taipei City (TW); Hsu-An Pan, Yilan County (TW); Yi-Zhe Liao, Taipei City (TW)

(21) Appl. No.: 18/217,661

(22) Filed: Jul. 3, 2023

(30) Foreign Application Priority Data
Apr. 6, 2023 (TW) 112112767

(51) Int. Cl. C09D 11/04; A61L 27/22; A61L 27/36

(52) U.S. Cl. CPC C09D 11/04; A61L 27/222; A61L 27/3633; A61L 27/3637

(57) ABSTRACT

A method for manufacturing a sterile bio-ink includes providing an extracellular matrix composition, and the extracellular matrix composition being in a liquid state; adding an animal-based gel and a plant-based gel that are freeze-dried, re-dissolved, and filtered to the extracellular matrix composition, and mixing the extracellular matrix composition, the animal-based gel, and the plant-based gel to obtain a gel mixture; and centrifuging and degassing the gel mixture to obtain the bio-ink.

### CROSS-REFERENCE TO RELATED PATENT APPLICATION

This application claims the benefit of priority to Taiwan Patent Application No. 112112767, filed on Apr. 6, 2023. The entire content of the above identified application is incorporated herein by reference.

Some references, which may include patents, patent applications and various publications, may be cited and discussed in the description of this disclosure. The citation and/or discussion of such references is provided merely to clarify the description of the present disclosure and is not an admission that any such reference is “prior art” to the disclosure described herein. All references cited and discussed in this specification are incorporated herein by reference in their entireties and to the same extent as if each reference was individually incorporated by reference.

### FIELD OF THE DISCLOSURE

The present disclosure relates to a method for manufacturing a bio-ink, and more particularly to a method for manufacturing a sterile bio-ink adopted in three-dimensional bio-printing of cells.

### BACKGROUND OF THE DISCLOSURE

Bio-printing is to print tissue organs or their analogs to replace animal experiments by using biomaterials for basic research on tissue organs to replace animal testing, and the bio-printed tissue organs are further expected to be used for human organ transplantation. To ensure the biocompatibility of bio-printed products, biomaterials are mostly based on gelatin or collagen, but it is difficult to control the softness or hardness of the bio-printed products using these materials, which affects the printability of a bio-ink.

In addition, nanocellulose and other thickening agents are usually added to a conventional ion-cured bio-ink to increase printability, so that the bio-ink becomes non-transparent, thus making the bio-printed product unsuitable for observation and use. In addition, conventional effective sterilization methods for solid and liquid products usually include physical sterilization and chemical sterilization. In order to ensure the physical properties and chemical composition of the bio-ink, the bio-ink is unable to be effectively sterilized by using conventional means such as radiation, high temperature, or ethylene oxide. Thus, there is a need for a bio-printing material that is sterile, has good biocompatibility, and maintains a certain degree of printability and transparency to meet the requirements in existing technologies.

Therefore, how to improve the printability, sterile property, and transparency of the bio-ink through improvements in manufacturing methods to overcome the aforementioned problems has become an issue to be addressed in the industry.

### SUMMARY OF THE DISCLOSURE

In response to the above-referenced technical inadequacies, the present disclosure provides a method for manufacturing a sterile bio-ink. The method includes: providing an extracellular matrix composition, and the extracellular matrix composition being in a liquid state; adding an animal-based gel and a plant-based gel that are freeze-dried, re-dissolved, and filtered to the extracellular matrix composition, and mixing the extracellular matrix composition, the animal-based gel, and the plant-based gel to obtain a gel mixture; and centrifuging and degassing the gel mixture to obtain the bio-ink.

In certain embodiments, the extracellular matrix composition is manufactured by the following steps: providing a gel; carrying out a crosslinking treatment, and the crosslinking treatment including adding a crosslinking agent to the gel to obtain a crosslinked gel; carrying out a cell culture, and the cell culture including implanting cells on the crosslinked gel and incubating the cells by adding a culture solution; carrying out a decrosslinking treatment, and the decrosslinking treatment including adding a decrosslinking agent to the crosslinked gel to obtain a decrosslinked mixture; and carrying out an extraction treatment, and the extraction treatment including adding a lysis enzyme to the decrosslinked mixture and filtering the decrosslinked mixture to obtain the extracellular matrix composition.

Therefore, in the method for manufacturing a sterile bio-ink provided by the present disclosure, by virtue of “the extracellular matrix composition being in a liquid state,” and “adding an animal-based gel and a plant-based gel that are freeze-dried, re-dissolved, and filtered to the extracellular matrix composition,” the bio-ink that is transparent and sterile can be manufactured, and the printability of the bio-ink can be maintained.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The described embodiments may be better understood by reference to the following description and the accompanying drawings, in which:

FIG. 1A is a histogram showing differences in cell viability when using different bio-ink products to carry out cell culture;

FIG. 1B is a histogram showing differences in cell multiplicity when using different bio-ink products to carry out cell culture;

FIG. 2A is a histogram showing changes in an average cell diameter in a cell culture that uses a bio-ink manufactured according to embodiments of the present disclosure;

FIG. 2B is a histogram showing changes in a maximum cell diameter in a cell culture that uses the bio-ink manufactured according to embodiments of the present disclosure;

FIG. 3 is a schematic view showing various stained cells undergone cellular differentiation in a cell culture that uses the bio-ink manufactured according to embodiments of the present disclosure;

FIG. 4A is a histogram showing changes in an area of a spheroid cell in a cell culture that uses the bio-ink manufactured according to embodiments of the present disclosure;

FIG. 4B is a histogram showing changes in a diameter of a spheroid cell in a cell culture that uses the bio-ink manufactured according to embodiments of the present disclosure; and

FIG. 5 is a schematic line chart of differences in light transmittance between embodiments and Comparative Examples according to embodiments of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

Embodiments of the present disclosure provide a method for manufacturing a sterile bio-ink. Specifically, embodiments of the present disclosure provide a method for manufacturing a sterile bio-ink that includes an extracellular matrix composition. Therefore, the method for manufacturing the sterile bio-ink includes providing an extracellular matrix composition. The extracellular matrix composition can be in a liquid state and can be directly mixed with other components without having to produce the extracellular matrix composition in advance, thereby reducing a manufacturing time of the bio-ink.

In addition, a content of the extracellular matrix composition in the bio-ink is from 92 wt % to 95 wt %. Therefore, compared to a bio-ink produced by using synthetic materials, the bio-ink of the present disclosure provides a more suitable growth environment for cells, and more effectively assists in cellular differentiation. In one embodiment of the present disclosure, the extracellular matrix composition is manufactured by steps as follows: providing a gel; carrying out a crosslinking treatment; carrying out a cell culture; carrying out a decrosslinking treatment; and carrying out an extraction treatment.

Specifically, the gel can be a sodium alginate having a concentration of from 3 vol % to 5 vol %. When a concentration of the sodium alginate is less than 3 vol %, a gel body formed after crosslinking has insufficient stiffness, and when the concentration of the sodium alginate is greater than 5 vol %, the sodium alginate becomes exceedingly stiff during crosslinking, so that it is difficult for the gel to have a smooth form. Therefore, when the concentration of the sodium alginate is from 3 vol % to 5 vol %, a stable carrying surface for cells can be provided after crosslinking.

Furthermore, a viscosity of the sodium alginate can be from 1 cP to 100 cP for the sodium alginate to be flatly disposed at a bottom of a container. In one embodiment of the present disclosure, a height of the gel is from 10 mm to 40 mm. When the height of the gel is less than 10 mm, the gel may crack during relevant operations, and if the height of the gel is greater than 40 mm, crosslinking reactions may be non-uniformly carried out in the gel.

In one embodiment of the present disclosure, nutritional components, such as vitamins and growth factors that are needed by the cells can be further added to the gel, so as to stimulate cell proliferation and cellular differentiation and increase a growth speed of the cells.

In the step of adding a crosslinking agent to the gel, the gel is changed from an original gel state to a jelly-like solid state to obtain a crosslinked gel. The crosslinking agent is a substance that bonds multiple linear molecules to each other so that the multiple linear molecules are crosslinked into a network structure. In the present disclosure, the crosslinking agent may contain metal ions, and a valence of the metal ions can be more than two. For example, the crosslinking agent may be calcium chloride, barium chloride, zinc chloride, calcium carbonate, calcium sulfate, or calcium lactate. In one preferred embodiment of the present disclosure, the crosslinking agent is from 5 vol % to 25 vol % of a calcium chloride solution, and the crosslinking volume ratio of sodium alginate to calcium chloride can be from 1 to 8.5, so as to achieve a better crosslinking effect.

In addition, duration for carrying out crosslinking can be from 5 minutes to 10 minutes. If the duration for carrying out crosslinking is less than 5 minutes, only the outside of the gel is cured, thus resulting in incomplete curing. On the other hand, it is not economical for the duration for carrying out crosslinking to be more than 10 minutes. The crosslinking temperature can be from 4° C. to 37° C. After the crosslinking process, the gel is rinsed twice with phosphate buffered saline (PBS) to remove residual crosslinking agent. It should be noted that the state of the cross-linked gel of the present disclosure will not be changed by changes in temperature.

Afterwards, the cells are implanted on the crosslinked gel and incubated with a culture solution. The cells used in this disclosure can be fibroblasts (e.g. 3T3 cells), mesenchymal stem cells (MSC), human and animal MSC, human hepatocellular carcinoma cells, epithelial cells, endothelial cells, or epidermal stem cells. The cell density for implantation is preferably from 51,000 cells/cm² to 85,000 cells/cm². It should be noted that the cell culture of the present disclosure is a single type cell culture, i.e., a single type of cell to be cultured is selected for cell culture.

In one embodiment of the present disclosure, the composition of the culture solution may include glycine, L-arginine hydrochloride, L-glutamine, L-isoleucine, L-leucine, L-methionine, L-phenylalanine, L-serine, L-threonine, L-tryptophan, L-valine, choline chloride, calcium pantothenate, niacinamide, pyridoxine hydrochloride, riboflavin, thiamine hydrochloride, and D-glucose.

In one preferred embodiment of the present disclosure, the culture solution can further include 50 μg/ml to 100 μg/ml of an ascorbic acid, so as to increase a growth number of the cells. In other words, a concentration of the ascorbic acid in the culture solution can be from 0.005% to 0.01%.

In one embodiment of the present disclosure, cells are cultured at an environment having a pH value of from 7.2 to 7.4, and having 5% to 10% of CO₂ for 3 days to 7 days. After the completion of cell culture, the culture solution is removed and the cells are rinsed for one time by using PBS. Before the decrosslinking treatment, the cells can undergo decellularization treatment by means of a decellularizing agent to facilitate the preparation of an extracellular matrix composition that is more biocompatible. In the present disclosure, the decellularizing agent may be a mixture of ammonium hydroxide (NH₄OH) and a non-ionic surfactant. For example, the non-ionic surfactant may be TRITON™ X-100, dodecyl maltoside, digitonin, tween 20, tween 80, etc.

In one preferred embodiment of the present disclosure, the decellularization treatment can include treating the cells with 20 mM of ammonium hydroxide and 0.5 vol % to 1 vol % of TRITON™ X-100 for 5 minutes to 15 minutes, and rinsing the cells twice with PBS. However, the above examples are only one possible embodiment and are not intended to limit the present disclosure.

Next, a decrosslinking agent is added to the crosslinked gel to obtain a decrosslinked mixture. In one embodiment of the present disclosure, the decrosslinking agent may be sodium citrate or ethylenediaminetetraacetic acid. For example, the decrosslinking treatment is adding 0.3 M to 0.6 M of sodium citrate to the crosslinked gel such that a content of sodium citrate is 1.3% to 2.6% of the gel, mixing the gel and sodium citrate for 1 hour to 3 hours, and reacting at 4° C. for 8 hours to 16 hours to complete the decrosslinking treatment of the gel. After the decrosslinking treatment, the gel is a decrosslinked mixture solution that is in a liquid state.

Finally, an extraction treatment is carried out on the decrosslinked mixture to obtain the extracellular matrix composition in a liquid state. Specifically, the extraction treatment is performed by filtering the decrosslinked mixture through a filter having a pore size of 0.22 μm. Preferably, a lysis enzyme can be added to the decrosslinked mixture and stirred for 3 hours to 6 hours before the extraction treatment to increase the extraction efficiency. For example, the lysis enzyme can be pepsin or collagenase. The amount of lysis enzyme used is from 0.4% to 1.2% of the decrosslinked mixture.

In one example of the present disclosure, in order to provide a suitable reaction environment for the lysis enzyme, the pH value of the decrosslinked mixture can be adjusted to be from 2 to 4 by adding 1 N to 3 N of hydrochloric acid (HCl). After a lysis treatment is completed, the pH value of the decrosslinked mixture can be adjusted to be from 7 to 8 by adding sodium hydroxide (NaOH) to carry out neutralization reaction. The decrosslinked mixture is then stirred for 1 hour before being filtered.

In one embodiment of the present disclosure, the extraction treatment may include a first extraction treatment and a second extraction treatment. In the first extraction treatment, the decrosslinked mixture is first filtered through a filter having a pore size of 10 μm to obtain a first filtrate. Afterwards, deionized water (ddH₂O) is added to the first filtrate and stirred for 10 minutes to 30 minutes, and then the second extraction treatment is carried out. The second extraction treatment is carried out by filtering the first filtrate by using a filter having a pore size of 0.22 μm to obtain an extracellular matrix composition (i.e., a decellularized extracellular matrix gel) having a higher purity. Preferably, the first extraction treatment and the second extraction treatment use negative pressure filtration to increase the efficiency of the extraction treatment.

It should be further noted that, the gel can be provided by a two-dimensional (2D) gel manufacturing process or a three-dimensional (3D) gel manufacturing process. In the 2D gel manufacturing process, the gel is laid flat on the bottom surface of the culture vessel so that the cells do not adhere directly to the bearing surface of the culture vessel. In the 3D gel manufacturing process, the gel can have a micron-sized spherical structure or a laminar printed structure. The 2D gel manufacturing process and the 3D gel manufacturing process are described below in more detail.

In one embodiment of the present disclosure, a 2D gel manufacturing process is used. Sodium alginate having a concentration of 4 vol % is spread on a petri dish so that a gel layer has a height of 20 mm, calcium chloride having a concentration of 10 vol % is added to carry out crosslinking for 5 minutes, and the gel layer is then rinsed twice with PBS to remove the residual crosslinking agent. Fibroblasts are implanted on the gel layer at a density of 51,000 cells/cm². After 5 days of culturing, the culture solution is removed from the gel layer. The gel layer is then rinsed once with PBS, treated with 20 mM of NH₄OH and 1 vol % of TRITON™ X-100 for 10 minutes, and rinsed twice with PBS. After adding 0.4 M of sodium citrate to the gel layer, the gel layer and the sodium citrate are put into a beaker, mixed for 2 hours and placed at 4° C. for 8 hours to complete the decrosslinking treatment. In the extraction treatment, the pH value of the mixture is adjusted to 2 and then 240 mg of pepsin is added in the beaker and stirred for 3 hours. The pH value of the mixture is then adjusted to 7 and stirred for 1 hour. Afterwards, the mixture is filtered by using a filter having a pore size of 10 μm under negative pressure. Finally, deionized water is added until a volume of the entire mixture is 240 ml and the mixture is then mixed and stirred for 10 minutes, and then the mixture is filtered by using a filter having a pore size of 0.22 μm under negative pressure to obtain the decellularized extracellular matrix (dECM) gel.

A difference between a Comparative Example and embodiments of the present disclosure is that in the Comparative Example, the fibroblasts are directly implanted on the petri dish at a density of 51,000 cells/cm² for cell culture, i.e., the cells directly adhere to the petri dish. After 5 days of culturing, the culture solution is removed from the gel layer. The gel layer is then rinsed once with PBS, treated with 20 mM of NH₄OH and 1 vol % of TRITON™ X-100 for 10 minutes, and rinsed twice with PBS. Afterwards, the petri dish is freeze-dried in a freeze-dryer for 24 hours, and then the freeze-dried cell powder is removed from the petri dish by scraping with a spatula for extraction treatment. In the extraction treatment, the pH value of the mixture is adjusted to 2 and 240 mg of pepsin is added and mixed for 3 hours, and then the pH value of the mixture is adjusted to 7 and the mixture is mixed for 1 hour. The mixture is then filtered by using a filter having a pore size of 0.22 μm under negative pressure to obtain the dECM gel.

Total protein concentrations of products of the preceding embodiments and the Comparative Example are measured by using the Bio-Rad protein assay kit, and the protein concentrations are further obtained by combining the reagent with the protein and measuring light absorbance value by using colorimetric analysis. The total protein concentration of the product in this embodiment of the present disclosure is 38.21±4.65 mg, and is higher than the total protein concentration of 19.00±4.58 mg of the Comparative Example. In addition, for the average amount of dECM produced per cell, after calculation, the average amount of dECM produced per cell in this embodiment of the present disclosure is 0.76±0.09 dECM/cells(ng), which is higher than 0.38±0.09 dECM/cells(ng) as in the Comparative Example. Therefore, in the present disclosure, the method for manufacturing a sterile bio-ink has higher productivity compared with the conventional method as shown by the Comparative Example.

Further, amounts of DNA residue of the embodiments of the present disclosure and that of the Comparative Example are compared. An amount of DNA residue in the dECM is determined by measuring the absorbance value of samples at a wavelength of 545 nm by using the dsDNA assay kit, and calculating the DNA content. The method for manufacturing a sterile bio-ink provided in the present disclosure includes a decellularization treatment that provides excellent decellularization while increasing yield, therefore resulting in a finished dECM product having almost no DNA residue.

It should be noted that the use of the dECM gel of the present disclosure in cell culture can contribute to cell growth. In petri dishes initially having 2×10⁵ fibroblasts, equal amounts of dECM gel produced in the 2D gel manufacturing process of the present disclosure and a dECM solution produced in the Comparative Example are respectively added and cultured for 24 hours. In the embodiment in which the dECM gel of the present disclosure is added, the fibroblasts are coated with the dECM gel of the present disclosure, and the cell number is increased by more than two times. In other words, the dECM gel produced in the 2D gel manufacturing process of the present disclosure has a higher concentration, and can assist the cells to proliferate rapidly.

In petri dishes initially having 2×10⁵ fibroblasts, equal amounts of dECM gel produced in the 2D gel manufacturing process of the present disclosure and the dECM solution produced in the Comparative Example are respectively added and cultured for 5 hours to observe cell adhesion and extension. The results show that the number of cell adhesion in the embodiment having the dECM gel of the present disclosure is significantly higher than the number of cell adhesion in the embodiment having the dECM gel of the Comparative Example, and the dECM gel of the present disclosure stabilizes the adhesion and growth of the cells. Furthermore, in the embodiment to which the dECM gel of the present disclosure is added, the cells have a larger extension area compared to the cells in the Comparative Example.

In another embodiment of the present disclosure, a 3D gel manufacturing process is used. The 3D gel manufacturing process is substantially the same as the aforementioned 2D gel manufacturing process. A difference between the two manufacturing processes is that, in the 2D gel manufacturing process, the sodium alginate is spread on the petri dish carrier surface; in one embodiment of the 3D gel manufacturing process, the gel can have a micron-sized spherical structure, and the size of the micron-sized spherical structure can be controlled to achieve the desired cell culture volume. The average particle size of the micron-sized spherical structure can range from 0.2 mm to 2 mm, preferably from 0.4 mm to 1.5 mm, and the particle size of each micron-sized spherical structure can be different, so as to improve the cell culture volume. In another embodiment of the 3D gel manufacturing process, the gel may have a laminar printed structure. However, the above examples are only one possible embodiment and are not intended to limit the scope of the present disclosure.

In one embodiment of the present disclosure, through the 3D gel manufacturing process, the gel is manufactured into micron-sized spherical structures having an average particle size of 0.4 mm. The total protein concentration of the product in this embodiment of the present disclosure is 73.24±2.64 mg, and is higher than the total protein concentration of 19.00±4.58 mg of the Comparative Example. In addition, for the average amount of dECM produced per cell, after calculation, the average amount of dECM produced per cell in this embodiment of the present disclosure is 1.46±0.05 dECM/cells(ng), which is higher than 0.38±0.09 dECM/cells(ng) as in the Comparative Example. Therefore, compared to the conventional method (i.e., the Comparative Example), the method for manufacturing a sterile bio-ink of the present disclosure has higher single cell yield, and the 3D gel manufacturing process has better performance than the 2D gel manufacturing process. Therefore, the method for manufacturing a sterile bio-ink of the present disclosure can effectively save operation time and obtain high yield of extracellular matrix composition. In addition, in the 3D gel manufacturing process, the yield can be further increased while maintaining excellent decellularization effect, so that the finished dECM product has almost no DNA residue.

Further, the method for manufacturing the sterile bio-ink of the present disclosure includes adding an animal-based gel and a plant-based gel to the aforementioned extracellular matrix composition and stirring the gel mixture at a temperature of from 40° C. to 55° C. The bio-ink of the present disclosure is obtained after centrifugation and degassing the gel mixture. Specifically, the animal-based gel and the plant-based gel that are freeze-dried, re-dissolved, and filtered are added to the extracellular matrix composition as a sterile liquid.

Further, the aforementioned sterile liquid is prepared from sterile animal-based gel powder and sterile plant-based gel powder. In the present disclosure, the sterile animal-based gel powder and the sterile plant-based gel powder are prepared by the following steps: adding animal-based gel material and plant-based gel material to deionized water to prepare a dilute solution with a concentration of 0.5 wt % to 2 wt %, so as to obtain an animal-based and plant-based gel dilute solution; the dilute solution is filtered and sterilized under negative pressure to obtain an animal-based and plant-based gel dilute solution that is filtered and sterilized; the animal-based and plant-based gel dilute solution that is filtered and sterilized is freeze-dried to obtain a sterile animal-based and plant-based gel powder.

In one embodiment of the present disclosure, the bio-ink contains from 2 wt % to 4 wt % of an animal-based gel that is selected from the group consisting of gelatin, gelatin methacrylamide, Pluronic F-127, collagen, chitosan, and hyaluronic acid. In one preferred embodiment of the present disclosure, the animal-based gel is gelatin.

In one embodiment of the present disclosure, the bio-ink contains from 3 wt % to 4 wt % of a plant-based gel that is selected from the group consisting of sodium alginate, agar, carboxymethyl cellulose, tragacanth gum, gum arabic, xanthan gum, pectin, guar gum, and carrageenans. In a preferred embodiment of the present disclosure, the plant-based gel is sodium alginate.

It is worth mentioning that when a weight ratio of the extracellular matrix composition, the plant-based gel, and the animal-based gel ranges from 100:3.5:2 to 100:3.5:4, the bio-ink that is produced is a transparent gel. Specifically, the bio-ink of the present disclosure can achieve a light transmission rate that is greater than or equal to 70%, and a viscosity of the bio-ink ranges from 1 Pa·s to 20,000 Pa·s. Therefore, the bio-ink of the present disclosure has good printability and can be used for printing a transparent printing product.

### EMBODIMENTS

In one embodiment of the present disclosure, 95 wt % of the extracellular matrix composition, 2 wt % of an animal-based gel and 3 wt % of a plant-based gel are mixed and stirred under a temperature of 50° C. for 2 hours, and the mixture is centrifuged and degassed at 1500 rpm for 10 minutes to obtain the bio-ink.

After mixing the cells with the bio-ink, the bio-ink containing the cells is filled into a 3 ml syringe of an inkjet printer (3DDISCOVERY™ of REGENHU™), and the bio-ink is printed out at a speed of 2 mm/s through a 22G bio-printing nozzle to produce a product with a size of 10 mm×10 mm and a thickness of 0.35 mm. The test results show that the bio-ink can be printed at a pressure of from 0.4 bar to 0.6 bar and has printability. The printed product is crosslinked with 0.73 vol % of calcium chloride for 5 minutes and then rinsed twice with PBS to remove residual crosslinking agent. A culture solution is further added to the printed product to carry out cell culture. The cells are cultured in an environment having a pH value of from 7.2 to 7.4 and a CO₂ concentration of from 5% to 10% for 3 days to 7 days to complete the cell culture. It is worth mentioning that, after the bio-ink of the present disclosure is mixed with the cells for printing, using 0.73 vol % of calcium chloride to perform crosslinking in the bio-ink for 5 minutes can meet the general requirements of cell culture using bio-ink and maintain the stability and transparency of the printed structure.

After the completion of 3D cell culture with the bio-ink of the present disclosure, the culture solution is removed and the cell is rinsed once with PBS, and 0.1 M to 0.3 M of sodium citrate is added so that the content of sodium citrate is 1.5 wt % to 4.5 wt % of the gel. The mixture is reacted at a temperature of from 20° C. to 37° C. for 5 minutes to 10 minutes to complete the decrosslinking of the gel, and complete cells or cell pellets cultured in the original gel can be obtained for subsequent applications (e.g., drug screening). In one embodiment of the present disclosure, the cell culture is followed by a decrosslinking step of adding 0.3 M of sodium citrate and allowing the mixture to react at a temperature of 25° C. for 5 minutes. It is worth mentioning that, after the cells and the bio-ink are mixed and printed out and the crosslinking step is completed, the decrosslinking step of adding 0.3 M of sodium citrate and carrying out reaction for 5 minutes can allow intact cell groups to be obtained without damaging the 3D structure of the cell group while satisfying the need for cell culture by using the bio-ink of the present disclosure.

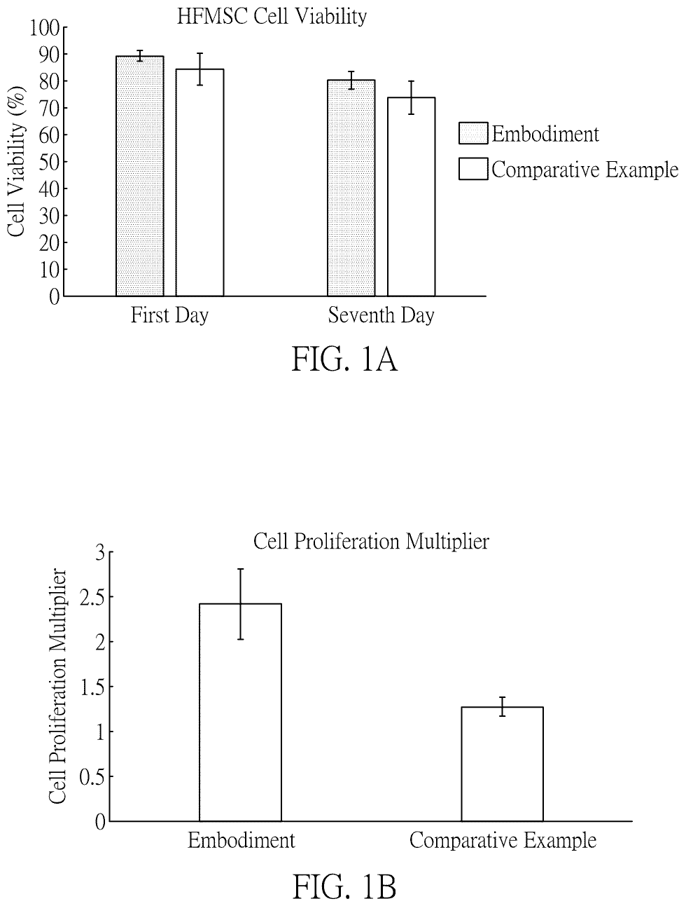

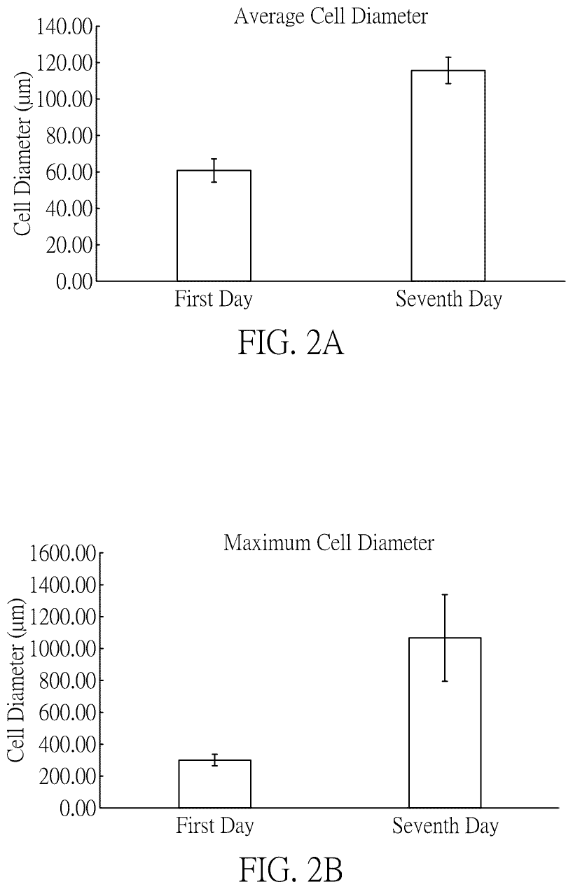

In one embodiment of the present disclosure, the printed product can be used to culture human mesenchymal stem cells (HFMSC). As shown in FIG. 1A, FIG. 1A is a histogram showing differences in cell viability when using different bio-ink products to carry out cell culture. For comparison, printed products having a same size are made using gelatin-based hydrogel (GBH) without extracellular matrix for cell culture. Cell viability is measured using the LIVE/DEAD™ cell imaging kit (produced by THERMO FISHER SCIENTIFIC™, Waltham, MA, US). As shown by the result in FIG. 1A, the cell viability of HFMSC cultured with the printed products printed with the bio-ink of the present disclosure is higher than 80% after 7 days, which is a higher cell viability compared to that of the Comparative Example.

FIG. 1B is a histogram showing differences in cell multiplicity when using different bio-ink products to carry out cell culture. The number of cells is measured with Cell Counting Kit-8 (CCK-8 produced by SIGMA-ALDRICH™, St. Louis, MO, USA) on a first day and a seventh day of cell culture, and the proliferation multiplier is calculated. Specifically, the original culture solution is removed and 100 μl of a new culture solution (containing 10% of CCK-8 reagent) is added, the mixture is placed at 37° C. for about 30 minutes, and the absorbance value is measured by using a spectrophotometer at a wavelength of 450 nm. As shown by the results of FIG. 1B, comparing with the cell proliferation rate of less than 1.5 times as in the Comparative Example, the cell proliferation rate of HFMSC cultured with the printed product of the bio-ink of the present disclosure is nearly 2.5 times after 7 days, and the bio-ink of the present disclosure is shown to be effective in increasing the cell growth rate.

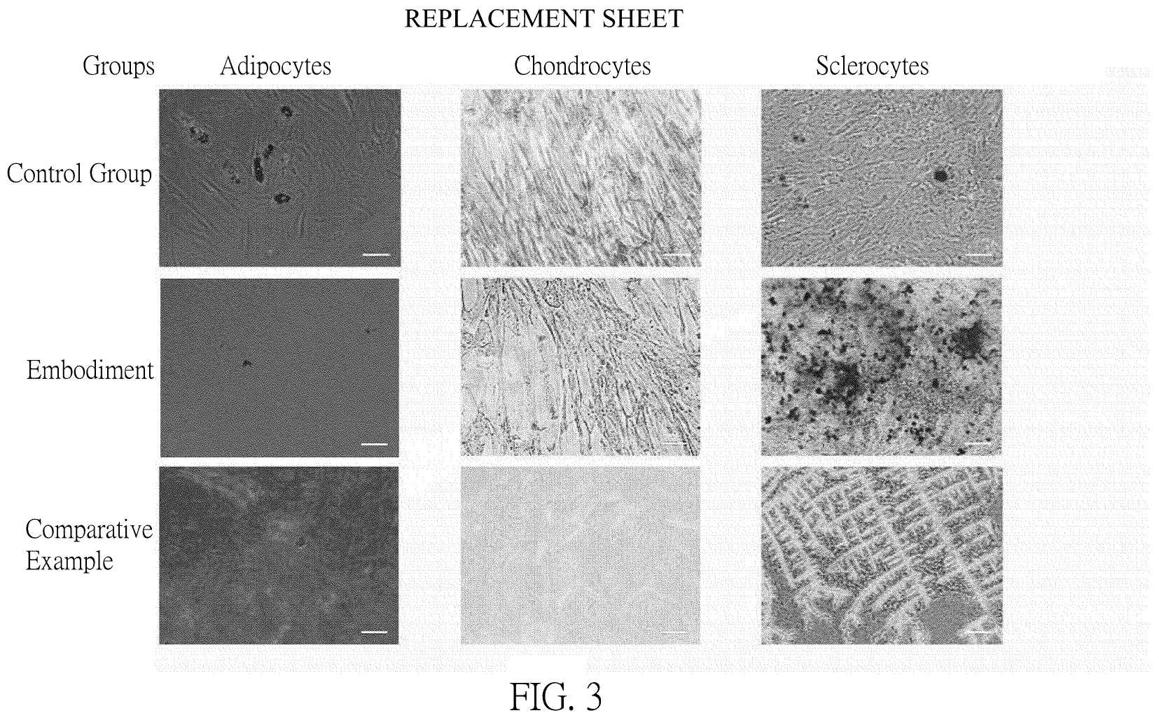

Since the bio-ink of the present disclosure provides a growth environment more suitable for maintaining the differentiation potential of stem cells, it is more effective in helping stem cells to undergo specified differentiation under specific conditions. In FIG. 3, in the control group, the cell culture of the HFMSC is directly carried out using culture solution. In the Embodiment, the cell culture of the HFMSC is carried out using the bio-ink of the present disclosure. In the Comparative Example, the cell culture of the HFMSC is carried out using hydrogel without extracellular matrix. As shown in FIG. 3, when the bio-ink of the present disclosure is used to culture the HFMSC, the HFMSC can differentiate into adipocytes, chondrocytes, and sclerocytes. In other words, the bio-ink of the present disclosure can enable the stem cells to successfully control the differentiation type in a 3D growth environment.

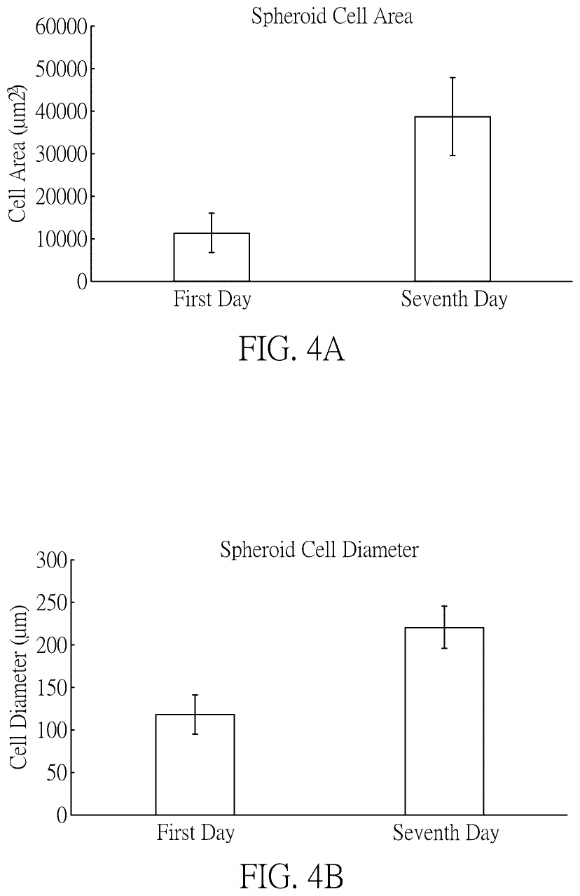

Furthermore, the bio-ink of the present disclosure can also be used to culture spheroid cells, such as Michigan Cancer Foundation-7 (MCF-7) cells. As shown in FIG. 4A and FIG. 4B, a spheroid cell area and a spheroid cell diameter increased significantly on a seventh day that MCF-7 cells are cultured using the bio-ink of the present disclosure. Therefore, the bio-ink manufactured by the method of the present disclosure can be used for in vitro simulation of tumor cells in 3D culture, which is beneficial for simulating the in vivo environment for cancer drug screening operations.

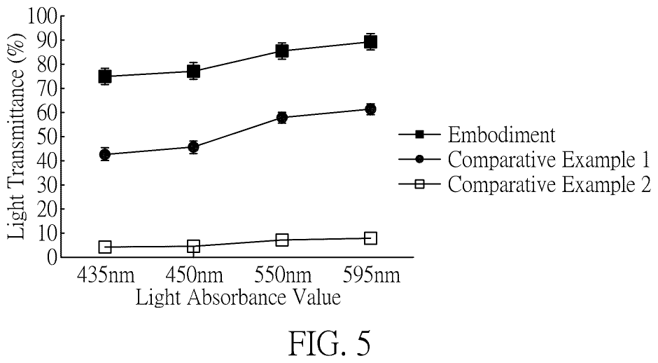

It is worth mentioning that, by a specific ratio between the extracellular matrix composition, the plant-based gel, and the animal-based gel, a transparent bio-ink can be obtained in the present disclosure. As shown in FIG. 5, a Comparative Example 1 is 6% of GBH and a Comparative Example 2 is a cellulose nanofiber hydrogel (CNH). As shown by the results of FIG. 5, the light transmission rate of the Comparative Example 1 is about from 40% to 60%, the light transmission rate of the Comparative Example 2 is less than 10%, and the bio-ink of the present disclosure can have a light transmission rate of more than 70%. Therefore, the bio-ink produced by the method of the present disclosure can be used to print transparent printing products while allowing the cells to be observed in real-time.

### Beneficial Effects of the Embodiments

In conclusion, in the method for manufacturing a sterile bio-ink provided by the present disclosure, by virtue of “the extracellular matrix composition being in a liquid state,” and “adding an animal-based gel and a plant-based gel that are freeze-dried, re-dissolved, and filtered to the extracellular matrix composition,” the bio-ink that is transparent and sterile can be manufactured, and the printability of the bio-ink can be maintained.

Furthermore, the extracellular matrix has poor gel kinetic properties, so that the accuracy of 3D bio-printing using the extracellular matrix is limited. The present disclosure provides a method for manufacturing a sterile bio-ink, which can produce a dECM gel having properties of a gel such as having a viscosity of from 1 cP to 100 cP, and components of the dECM gel include proteins, cytokines, growth factors, etc. Compared to the dECM solution produced by using the conventional method, the dECM gel is more suitable for manufacturing of bio-ink.

Further, the transparency of the bio-ink depends on the ratio of plant-based gels. In a preferred embodiment of the present disclosure, the weight ratio of the extracellular matrix composition, the plant-based gel, and the animal-based gel in the bio-ink is from 100:3.5:2 to 100:3.5:4. If the ratio of plant-based gels is higher than 3.5, the transparency of the bio-ink will be reduced to be from 40% to 50%. In addition, the printability of the bio-ink depends on the ratio of the animal-based gel. If the weight ratio of the animal-based gel is less than 2, the bio-ink will be in a liquid state and cannot achieve the effect of layered printing, and if the weight ratio of the animal-based gel is greater than 4, the bio-ink will turn into a solid state and easily blocks the printing nozzle, making it impossible to continue printing.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A method for manufacturing a sterile bio-ink, comprising:
providing an extracellular matrix composition, wherein the extracellular matrix composition is in a liquid state;
adding an animal-based gel and a plant-based gel that are freeze-dried, re-dissolved, and filtered to the extracellular matrix composition, and mixing the extracellular matrix composition, the animal-based gel, and the plant-based gel to obtain a gel mixture; and
centrifuging and degassing the gel mixture to obtain the bio-ink.

2. The method according to claim 1, wherein the extracellular matrix composition is manufactured by the following steps:
providing a gel;
carrying out a crosslinking treatment, wherein the crosslinking treatment includes adding a crosslinking agent to the gel to obtain a crosslinked gel;
carrying out a cell culture, wherein the cell culture includes implanting cells on the crosslinked gel and incubating the cells by adding a culture solution;
carrying out a decrosslinking treatment, wherein the decrosslinking treatment includes adding a decrosslinking agent to the crosslinked gel to obtain a decrosslinked mixture; and
carrying out an extraction treatment, wherein the extraction treatment includes adding a lysis enzyme to the decrosslinked mixture and filtering the decrosslinked mixture to obtain the extracellular matrix composition.

3. The method according to claim 1, wherein a content of the extracellular matrix composition in the bio-ink is from 92 wt % to 95 wt %.

4. The method according to claim 1, wherein the animal-based gel is selected from the group consisting of gelatin, gelatin methacrylamide, Pluronic F-127, collagen, chitosan, and hyaluronic acid.

5. The method according to claim 1, wherein the plant-based gel is selected from the group consisting of sodium alginate, agar, carboxymethyl cellulose, tragacanth gum, gum arabic, xanthan gum, pectin, guar gum, and carrageenans.

6. The method according to claim 1, wherein the bio-ink contains from 3 wt % to 4 wt % of the plant-based gel.

7. The method according to claim 1, wherein the bio-ink contains from 2 wt % to 4 wt % of the animal-based gel.

8. The method according to claim 1, wherein a weight ratio of the extracellular matrix composition, the plant-based gel, and the animal-based gel ranges from 100:3.5:2 to 100:3.5:4.

9. The method according to claim 1, wherein the bio-ink has a light transmission rate that is greater than or equal to 70%.

10. The method according to claim 1, wherein a viscosity of the bio-ink ranges from 1 Pa·s to 20,000 Pa·s.
